(19) United States
(12) Patent Application Publication
(10) Pub. No.: US 2022/0378814 A1
(43) Pub. Date: Dec. 1, 2022

(54) COMPOSITION OF PHYTONUTRIENTS FOR OBESITY PREVENTION

(71) Applicant: ROSEMAN UNIVERSITY OF HEALTH SCIENCES, Las Vegas, NV (US)

(72) Inventors: Thuc T. LE, Las Vegas, NV (US); Yasuyo Urasaki, Las Vegas, NV (US)

(21) Appl. No.: 17/741,157

(22) Filed: May 10, 2022

Related U.S. Application Data
(60) Provisional application No. 63/186,993, filed on May 11, 2021.

(51) Int. Cl. A61K 31/7048; A61P 3/02; A61K 31/47; A61P 3/06; A61K 31/05; A61K 31/353; A61K 31/715

(52) U.S. Cl. CPC A61K 31/7048; A61P 3/02; A61K 31/47; A61P 3/06; A61K 31/05; A61K 31/353; A61K 31/715

(57) ABSTRACT

The present disclosure relates to compositions of phytonutrients and methods of treating obesity by administering these compositions to subjects in need thereof. The compositions described herein are rationally designed compositions of phytonutrients that interfere with fat cell differentiation, a process commonly known as “adipogenesis”, to prevent weight gain and improve glycemic control. The phytonutrients are rationally combined based on their complementary effects on the expression level of six adipogenic biomarker proteins. Exemplary compositions can include one or more of berberine, luteolin, resveratrol, fisetin, quercetin, fucoidan, epigallocatechin gallate (EGCG), hesperidin, or curcumin.

### CROSS-REFERENCE TO RELATED APPLICATIONS

The present application claims the benefit of U.S. Provisional Patent Application 63/186,993, filed May 11, 2021.

### BACKGROUND

The current obesity epidemic presents a major challenge to global health management. In the United States, the prevalence of obesity in adults was 42.4% in 2017-2018 [1]. The estimate of healthcare cost attributable to obesity was $190 billion in 2013, or approximately 21% of US healthcare expenditure [2]. The World Health Organization estimated that approximately 650 million adults were obese worldwide in 2016. Obesity is an established risk factor for the development of type 2 diabetes mellitus and chronic inflammatory diseases, such as dyslipidemia, non-alcoholic fatty liver disease, hypertension, coronary heart disease, stroke, rheumatoid arthritis, and certain cancers [3]. Weight loss is clearly the most obvious strategy for the prevention of obesity and associated diseases. However, achieving weight loss by means of lifestyle intervention has proven to be challenging for many patients to maintain in the long term. Alternative intervention strategies include pharmacotherapy to restrict caloric intake for weight management or glycemic control for diabetes management [4]. Two prevalent pharmacotherapy types are being pursued: those developed for glycemic control that might lead to weight gain, and those developed for weight management that improve glycemic control. Approved anti-obesity and antidiabetic drugs by the United States Food and Drug Administration have proven to be moderately beneficial although they are often associated with adverse side effects, which limit their long-term usage [5]. Clearly, there is still an unmet demand for an effective and safe intervention that is capable of long-term management of obesity and associated medical conditions.

### BRIEF DESCRIPTION OF THE DRAWINGS

FIG. 1A is a Western blot showing the expression level of PPARγ in preadipocytes (d0), differentiating adipocytes on day sixth post-differentiation (d6), and differentiating adipocytes on day sixth post-differentiation in the presence of phytonutrient C1. Expression level of PPARγ was measured with capillary Western immunoassays. β-actin served as a loading control.

FIG. 1B is a Western blot showing expression level of PPARγ in preadipocytes (d0), differentiating adipocytes on day sixth post-differentiation (d6), and differentiating adipocytes on day sixth post-differentiation in the presence of phytonutrient C2. Expression level measurement and loading control were as for FIG. 1A.

FIG. 1C is a Western blot showing expression level of PPARγ in preadipocytes (d0), differentiating adipocytes on day sixth post-differentiation (d6), and differentiating adipocytes on day sixth post-differentiation in the presence of phytonutrient C3. Expression level measurement and loading control were as for FIG. 1A.

FIG. 1D is a Western blot showing expression level of PPARγ in preadipocytes (d0), differentiating adipocytes on day sixth post-differentiation (d6), and differentiating adipocytes on day sixth post-differentiation in the presence of phytonutrient C4. Expression level measurement and loading control were as for FIG. 1A.

FIG. 1E is a Western blot showing expression level of PPARγ in preadipocytes (d0), differentiating adipocytes on day sixth post-differentiation (d6), and differentiating adipocytes on day sixth post-differentiation in the presence of phytonutrient C5. Expression level measurement and loading control were as for FIG. 1A.

FIG. 1F is a Western blot showing expression level of PPARγ in preadipocytes (d0), differentiating adipocytes on day sixth post-differentiation (d6), and differentiating adipocytes on day sixth post-differentiation in the presence of phytonutrient C6. Expression level measurement and loading control were as for FIG. 1A.

FIG. 1G is a Western blot showing expression level of PPARγ in preadipocytes (d0), differentiating adipocytes on day sixth post-differentiation (d6), and differentiating adipocytes on day sixth post-differentiation in the presence of phytonutrient C7. Expression level measurement and loading control were as for FIG. 1A.

FIG. 1H is a Western blot showing expression level of PPARγ in preadipocytes (d0), differentiating adipocytes on day sixth post-differentiation (d6), and differentiating adipocytes on day sixth post-differentiation in the presence of phytonutrient C8. Expression level measurement and loading control were as for FIG. 1A.

FIG. 1I is a Western blot showing expression level of PPARγ in preadipocytes (d0), differentiating adipocytes on day sixth post-differentiation (d6), and differentiating adipocytes on day sixth post-differentiation in the presence of phytonutrient C9. Expression level measurement and loading control were as for FIG. 1A.

FIG. 1J is a Western blot showing expression level of PPARγ in preadipocytes (d0), differentiating adipocytes on day sixth post-differentiation (d6), and differentiating adipocytes on day sixth post-differentiation in the presence of formulation F1, containing phytonutrients C1-C9. Expression level measurement and loading control were as for FIG. 1A.

FIG. 2A is a Western blot showing expression level of SREBP1c in preadipocytes (d0), differentiating adipocytes on day sixth post-differentiation (d6), and differentiating adipocytes on day sixth post-differentiation in the presence of phytonutrient C1. Expression level of SREBP1c was measured with capillary Western immunoassays. HSP60 served as a loading control.

FIG. 2B is a Western blot showing expression level of SREBP1c in preadipocytes (d0), differentiating adipocytes on day sixth post-differentiation (d6), and differentiating adipocytes on day sixth post-differentiation in the presence of phytonutrient C2. Expression level measurement and loading control were as for FIG. 2A.

FIG. 2C is a Western blot showing expression level of SREBP1c in preadipocytes (d0), differentiating adipocytes on day sixth post-differentiation (d6), and differentiating adipocytes on day sixth post-differentiation in the presence of phytonutrient C3. Expression level measurement and loading control were as for FIG. 2A.

FIG. 2D is a Western blot showing expression level of SREBP1c in preadipocytes (d0), differentiating adipocytes on day sixth post-differentiation (d6), and differentiating adipocytes on day sixth post-differentiation in the presence of phytonutrient C4. Expression level measurement and loading control were as for FIG. 2A.

FIG. 2E is a Western blot showing expression level of SREBP1c in preadipocytes (d0), differentiating adipocytes on day sixth post-differentiation (d6), and differentiating adipocytes on day sixth post-differentiation in the presence of phytonutrient C5. Expression level measurement and loading control were as for FIG. 2A.

FIG. 2F is a Western blot showing expression level of SREBP1c in preadipocytes (d0), differentiating adipocytes on day sixth post-differentiation (d6), and differentiating adipocytes on day sixth post-differentiation in the presence of phytonutrient C6. Expression level measurement and loading control were as for FIG. 2A.

FIG. 2G is a Western blot showing expression level of SREBP1c in preadipocytes (d0), differentiating adipocytes on day sixth post-differentiation (d6), and differentiating adipocytes on day sixth post-differentiation in the presence of phytonutrient C7. Expression level measurement and loading control were as for FIG. 2A.

FIG. 2H is a Western blot showing expression level of SREBP1c in preadipocytes (d0), differentiating adipocytes on day sixth post-differentiation (d6), and differentiating adipocytes on day sixth post-differentiation in the presence of phytonutrient C8. Expression level measurement and loading control were as for FIG. 2A.

FIG. 2I is a Western blot showing expression level of SREBP1c in preadipocytes (d0), differentiating adipocytes on day sixth post-differentiation (d6), and differentiating adipocytes on day sixth post-differentiation in the presence of phytonutrient C9. Expression level measurement and loading control were as for FIG. 2A.

FIG. 2J is a Western blot showing expression level of SREBP1c in preadipocytes (d0), differentiating adipocytes on day sixth post-differentiation (d6), and differentiating adipocytes on day sixth post-differentiation in the presence of formulation F1 comprising phytonutrients C1-C9. Expression level measurement and loading control were as for FIG. 2A.

FIG. 3A is a Western blot showing expression level of FASN in preadipocytes (d0), differentiating adipocytes on day sixth post-differentiation (d6), and differentiating adipocytes on day sixth post-differentiation in the presence of phytonutrient C1. Expression level of FASN was measured with capillary Western immunoassays. β-actin served as a loading control.

FIG. 3B is a Western blot showing expression level of FASN in preadipocytes (d0), differentiating adipocytes on day sixth post-differentiation (d6), and differentiating adipocytes on day sixth post-differentiation in the presence of phytonutrient C2. Expression level measurement and loading control were as for FIG. 3A.

FIG. 3C is a Western blot showing expression level of FASN in preadipocytes (d0), differentiating adipocytes on day sixth post-differentiation (d6), and differentiating adipocytes on day sixth post-differentiation in the presence of phytonutrient C3. Expression level measurement and loading control were as for FIG. 3A.

FIG. 3D is a Western blot showing expression level of FASN in preadipocytes (d0), differentiating adipocytes on day sixth post-differentiation (d6), and differentiating adipocytes on day sixth post-differentiation in the presence of phytonutrient C4. Expression level measurement and loading control were as for FIG. 3A.

FIG. 3E is a Western blot showing expression level of FASN in preadipocytes (d0), differentiating adipocytes on day sixth post-differentiation (d6), and differentiating adipocytes on day sixth post-differentiation in the presence of phytonutrient C5. Expression level measurement and loading control were as for FIG. 3A.

FIG. 3F is a Western blot showing expression level of FASN in preadipocytes (d0), differentiating adipocytes on day sixth post-differentiation (d6), and differentiating adipocytes on day sixth post-differentiation in the presence of phytonutrient C6. Expression level measurement and loading control were as for FIG. 3A.

FIG. 3G is a Western blot showing expression level of FASN in preadipocytes (d0), differentiating adipocytes on day sixth post-differentiation (d6), and differentiating adipocytes on day sixth post-differentiation in the presence of phytonutrient C7. Expression level measurement and loading control were as for FIG. 3A.

FIG. 3H is a Western blot showing expression level of FASN in preadipocytes (d0), differentiating adipocytes on day sixth post-differentiation (d6), and differentiating adipocytes on day sixth post-differentiation in the presence of phytonutrient C8. Expression level measurement and loading control were as for FIG. 3A.

FIG. 3I is a Western blot showing expression level of FASN in preadipocytes (d0), differentiating adipocytes on day sixth post-differentiation (d6), and differentiating adipocytes on day sixth post-differentiation in the presence of phytonutrient C9. Expression level measurement and loading control were as for FIG. 3A.

FIG. 3J is a Western blot showing expression level of FASN in preadipocytes (d0), differentiating adipocytes on day sixth post-differentiation (d6), and differentiating adipocytes on day sixth post-differentiation in the presence of formulation F1 containing phytonutrients C1-C9. Expression level measurement and loading control were as for FIG. 3A.

FIG. 4A is a Western blot showing expression level of PLIN1 in preadipocytes (d0), differentiating adipocytes on day sixth post-differentiation (d6), and differentiating adipocytes on day sixth post-differentiation in the presence of phytonutrient C1. Expression level of PLIN1 was measured with capillary Western immunoassays. β-actin served as a loading control.

FIG. 4B is a Western blot showing expression level of PLIN1 in preadipocytes (d0), differentiating adipocytes on day sixth post-differentiation (d6), and differentiating adipocytes on day sixth post-differentiation in the presence of phytonutrient C2. Expression level measurement and loading control were as for FIG. 4A.

FIG. 4C is a Western blot showing expression level of PLIN1 in preadipocytes (d0), differentiating adipocytes on day sixth post-differentiation (d6), and differentiating adipocytes on day sixth post-differentiation in the presence of phytonutrient C3. Expression level measurement and loading control were as for FIG. 4A.

FIG. 4D is a Western blot showing expression level of PLIN1 in preadipocytes (d0), differentiating adipocytes on day sixth post-differentiation (d6), and differentiating adipocytes on day sixth post-differentiation in the presence of phytonutrient C4. Expression level measurement and loading control were as for FIG. 4A.

FIG. 4E is a Western blot showing expression level of PLIN1 in preadipocytes (d0), differentiating adipocytes on day sixth post-differentiation (d6), and differentiating adipocytes on day sixth post-differentiation in the presence of phytonutrient C5. Expression level measurement and loading control were as for FIG. 4A.

FIG. 4F is a Western blot showing expression level of PLIN1 in preadipocytes (d0), differentiating adipocytes on day sixth post-differentiation (d6), and differentiating adipocytes on day sixth post-differentiation in the presence of phytonutrient C6. Expression level measurement and loading control were as for FIG. 4A.

FIG. 4G is a Western blot showing expression level of PLIN1 in preadipocytes (d0), differentiating adipocytes on day sixth post-differentiation (d6), and differentiating adipocytes on day sixth post-differentiation in the presence of phytonutrient C7. Expression level measurement and loading control were as for FIG. 4A.

FIG. 4H is a Western blot showing expression level of PLIN1 in preadipocytes (d0), differentiating adipocytes on day sixth post-differentiation (d6), and differentiating adipocytes on day sixth post-differentiation in the presence of phytonutrient C8. Expression level measurement and loading control were as for FIG. 4A.

FIG. 4I is a Western blot showing expression level of PLIN1 in preadipocytes (d0), differentiating adipocytes on day sixth post-differentiation (d6), and differentiating adipocytes on day sixth post-differentiation in the presence of phytonutrient C9. Expression level measurement and loading control were as for FIG. 4A.

FIG. 4J is a Western blot showing expression level of PLIN1 in preadipocytes (d0), differentiating adipocytes on day sixth post-differentiation (d6), and differentiating adipocytes on day sixth post-differentiation in the presence of formulation F1, comprising phytonutrients C1-C9. Expression level measurement and loading control were as for FIG. 4A.

FIG. 5A is a capillary isoelectric focusing immunoassay (cIEF) graph showing expression level of FABP4 in preadipocytes (d0), differentiating adipocytes on day sixth post-differentiation (d6), and differentiating adipocytes on day sixth post-differentiation in the presence of phytonutrient C1. Expression level of FABP4 was measured with capillary isoelectric focusing immunoassays. HSP70 served as a loading control (not shown).

FIG. 5B is a cIEF graph showing expression level of FABP4 in preadipocytes (d0), differentiating adipocytes on day sixth post-differentiation (d6), and differentiating adipocytes on day sixth post-differentiation in the presence of phytonutrient C2. Expression level was measured and loading control were as for FIG. 5A.

FIG. 5C is a cIEF graph showing expression level of FABP4 in preadipocytes (d0), differentiating adipocytes on day sixth post-differentiation (d6), and differentiating adipocytes on day sixth post-differentiation in the presence of phytonutrient C3. Expression level was measured and loading control were as for FIG. 5A.

FIG. 5D is a cIEF graph showing expression level of FABP4 in preadipocytes (d0), differentiating adipocytes on day sixth post-differentiation (d6), and differentiating adipocytes on day sixth post-differentiation in the presence of phytonutrient C4. Expression level was measured and loading control were as for FIG. 5A.

FIG. 5E is a cIEF graph showing expression level of FABP4 in preadipocytes (d0), differentiating adipocytes on day sixth post-differentiation (d6), and differentiating adipocytes on day sixth post-differentiation in the presence of phytonutrient C5. Expression level was measured and loading control were as for FIG. 5A.

FIG. 5F is a cIEF graph showing expression level of FABP4 in preadipocytes (d0), differentiating adipocytes on day sixth post-differentiation (d6), and differentiating adipocytes on day sixth post-differentiation in the presence of phytonutrient C6. Expression level was measured and loading control were as for FIG. 5A.

FIG. 5G is a cIEF graph showing expression level of FABP4 in preadipocytes (d0), differentiating adipocytes on day sixth post-differentiation (d6), and differentiating adipocytes on day sixth post-differentiation in the presence of phytonutrient C7. Expression level was measured and loading control were as for FIG. 5A.

FIG. 5H is a cIEF graph showing expression level of FABP4 in preadipocytes (d0), differentiating adipocytes on day sixth post-differentiation (d6), and differentiating adipocytes on day sixth post-differentiation in the presence of phytonutrient C8. Expression level was measured and loading control were as for FIG. 5A.

FIG. 5I is a cIEF graph showing expression level of FABP4 in preadipocytes (d0), differentiating adipocytes on day sixth post-differentiation (d6), and differentiating adipocytes on day sixth post-differentiation in the presence of phytonutrient C9. Expression level was measured and loading control were as for FIG. 5A.

FIG. 5J is a cIEF graph showing expression level of FABP4 in preadipocytes (d0), differentiating adipocytes on day sixth post-differentiation (d6), and differentiating adipocytes on day sixth post-differentiation in the presence of formulation F1 comprising phytonutrients C1-C9. Expression level was measured and loading control were as for FIG. 5A.

FIG. 6A is a Western blot showing expression level of β-catenin in preadipocytes (d0), differentiating adipocytes on day sixth post-differentiation (d6), and differentiating adipocytes on day sixth post-differentiation in the presence of phytonutrient C1. Expression level of β-catenin was measured with capillary Western immunoassays and presented graphically as chemiluminescence versus isoelectric points (pI). HSP60 served as a loading control.

FIG. 6B is a Western blot showing expression level of β-catenin in preadipocytes (d0), differentiating adipocytes on day sixth post-differentiation (d6), and differentiating adipocytes on day sixth post-differentiation in the presence of phytonutrient C2. Expression level measurement and loading control were as for FIG. 6A.

FIG. 6C is a Western blot showing expression level of β-catenin in preadipocytes (d0), differentiating adipocytes on day sixth post-differentiation (d6), and differentiating adipocytes on day sixth post-differentiation in the presence of phytonutrient C3. Expression level measurement and loading control were as for FIG. 6A.

FIG. 6D is a Western blot showing expression level of β-catenin in preadipocytes (d0), differentiating adipocytes on day sixth post-differentiation (d6), and differentiating adipocytes on day sixth post-differentiation in the presence of phytonutrient C4. Expression level measurement and loading control were as for FIG. 6A.

FIG. 6E is a Western blot showing expression level of β-catenin in preadipocytes (d0), differentiating adipocytes on day sixth post-differentiation (d6), and differentiating adipocytes on day sixth post-differentiation in the presence of phytonutrient C5. Expression level measurement and loading control were as for FIG. 6A.

FIG. 6F is a Western blot showing expression level of β-catenin in preadipocytes (d0), differentiating adipocytes on day sixth post-differentiation (d6), and differentiating adipocytes on day sixth post-differentiation in the presence of phytonutrient C6. Expression level measurement and loading control were as for FIG. 6A.

FIG. 6G is a Western blot showing expression level of β-catenin in preadipocytes (d0), differentiating adipocytes on day sixth post-differentiation (d6), and differentiating adipocytes on day sixth post-differentiation in the presence of phytonutrient C7. Expression level measurement and loading control were as for FIG. 6A.

FIG. 6H is a Western blot showing expression level of β-catenin in preadipocytes (d0), differentiating adipocytes on day sixth post-differentiation (d6), and differentiating adipocytes on day sixth post-differentiation in the presence of phytonutrient C8. Expression level measurement and loading control were as for FIG. 6A.

FIG. 6I is a Western blot showing expression level of β-catenin in preadipocytes (d0), differentiating adipocytes on day sixth post-differentiation (d6), and differentiating adipocytes on day sixth post-differentiation in the presence of phytonutrient C9. Expression level measurement and loading control were as for FIG. 6A.

FIG. 6J is a Western blot showing expression level of β-catenin in preadipocytes (d0), differentiating adipocytes on day sixth post-differentiation (d6), and differentiating adipocytes on day sixth post-differentiation in the presence of formulation F1 comprising phytonutrients C1-C9. Expression level measurement and loading control were as for FIG. 6A.

FIG. 7 shows cytoplasmic lipid droplet accumulation over time as a function of individual or combination of phytonutrients. First row: Preadipocytes were maintained in growth media. Second row: Differentiating adipocytes were induced with complete differentiation media on day 0. Third row: Differentiating adipocytes were induced with complete differentiation media supplemented with C5 on day 0. Fourth row: Differentiating adipocytes were induced with complete differentiation media supplemented with C2 & C5 on day 0. Fifth row: Differentiating adipocytes were induced with complete differentiation media supplemented with C1, C2, C3, & C5 on day 0. Sixth row: Differentiating adipocytes were induced with complete differentiation media supplemented with F1 on day 0. Day sixth, eighth, tenth, and fourteenth post-differentiation are labeled as d6, d8, d10, and d14, respectively. Cytoplasmic lipid droplets are white particles or blobs under phase-contrast microscopy.

FIG. 8A presents high-resolution images of cytoplasmic lipid droplet accumulation, specifically, brightfield images of preadipocytes (d0), differentiating adipocytes on 14^{th} day post-differentiation (d14), and differentiating adipocytes on 14^{th} day post-differentiation in the presence of F1 from day 0 to day 6 (d14, F1). Cells were fixed and stained for lipid droplets with oil Red O (red) and nuclei with hematoxylin (blue). Cells were visualized with brightfield microscopy.

FIG. 8B presents high-resolution images of cytoplasmic lipid droplet accumulation, specifically, confocal fluorescence images of preadipocytes (d0), differentiating adipocytes on 14^{th} day post-differentiation (d14), and differentiating adipocytes on 14^{th} day post-differentiation in the presence of F1 from day 0 to day 6 (d14, F1). Cells were fixed and stained for actin (green), lipid droplets (pink), nucleus (blue), and tubulin (red). Cells were visualized with multicolor confocal fluorescence microscopy.

FIG. 9A shows F1 reduces weight gain in diet-induced obesity (DIO) mice. Specifically, the figure shows average bodyweight as a function of time on specified diets of three group of mice: lean diet, high-fat diet, and high-fat diet supplemented with F1.

FIG. 9B shows F1 reduces weight gain in DIO mice, as demonstrated by average bodyweight at 17^{th} week as a function of mice group on specified diets. LD: lean diet; HFD: high-fat diet; HFD+F1: high-fat diet supplemented with F1.

FIG. 9C shows F1 reduces weight gain in DIO mice, as demonstrated by average rate of weight gain in grams per week (g/w) as a function of mice group on specified diets. The error bars indicate the standard deviations of 40 mice per animal group. The asterisks indicate statistical significance, which was calculated with Student's t-test and thresholded at p≤0.01 versus the high-fat diet group.

FIG. 10A shows F1 improves glucose tolerance in DIO mice, as demonstrated by blood glucose level as a function of time post injection.

FIG. 10B shows F1 improves glucose tolerance in DIO mice, as demonstrated by fold change in blood glucose level as a function of time post injection. The error bars indicate the standard deviations of 40 mice per animal group.

FIG. 11A shows F1 reduces blood lipids in DIO mice, as demonstrated by blood triglyceride level as a function of animal group on specified diets. LD: lean diet; HFD: high-fat diet; HFD+F1: high-fat diet supplemented with F1. Blood samples terminally collected after 17 weeks on specified diets were used for measurement of triglyceride and cholesterol. The error bars indicate the standard deviations of 40 mice per animal group. The asterisks indicate statistical significance, which was calculated with Student's t-test and thresholded at p≤0.01 versus the high-fat diet group.

FIG. 11B shows F1 reduces blood lipids in DIO mice, as demonstrated by blood cholesterol level as a function of animal group on specified diets. Diets, sample collection, and statistical measures were as for FIG. 11A.

FIG. 12A shows F1 reduces blood low-density lipoprotein (LDL) cholesterol in DIO mice, as demonstrated by blood high-density lipoprotein (HDL) cholesterol level as a function of animal group on specified diets. LD: lean diet; HFD: high-fat diet; HFD+F1: high-fat diet supplemented with F1. Blood samples were terminally collected after 17 weeks on specified diets. The error bars indicate the standard deviations of 40 mice per animal group. The asterisks indicate statistical significance, which was calculated with Student's t-test and thresholded at p≤0.01 versus the high-fat diet group.

FIG. 12B shows F1 reduces blood LDL cholesterol in DIO mice, as demonstrated by blood LDL cholesterol level as a function of animal group on specified diets. Diets, sample collection, and statistical measures were as for FIG. 12A.

FIG. 13A shows F1 suppresses liver steatosis and reduces liver weight in DIO mice, as demonstrated by H&E histology of liver tissues collected from three animal groups on specified diets. Images were acquired with brightfield microscopy. Lipid droplets are indicated as white dots. Diets: LD: lean diet; HFD: high-fat diet; HFD+F1: high-fat diet supplemented with F1. The error bars indicate the standard deviations of 40 mice per animal group. The asterisks indicate statistical significance, which was calculated with Student's t-test and thresholded at p≤0.01 versus the high-fat diet group. Liver tissues were terminally collected after 17 weeks on specified diets.

FIG. 13B shows F1 suppresses liver steatosis and reduces liver weight in DIO mice, as demonstrated by liver weight as a function of animal groups on specified diets. Diets, sample collection, and statistical measures were as for FIG. 13A.

FIG. 14A shows F1 reduces visceral adiposity in DIO mice, as demonstrated by H&E histology of visceral adipose tissues collected from three animal groups on specified diets. LD: lean diet; HFD: high-fat diet; HFD+F1: high-fat diet supplemented with F1. The error bars indicate the standard deviations of 40 mice per animal group. The asterisks indicate statistical significance, which was calculated with Student's t-test and threshold at p≤0.01 versus the high-fat diet group. Visceral adipose tissues were terminally collected after 17 weeks on specified diets.

FIG. 14B shows F1 reduces visceral adiposity in DIO mice, as demonstrated by visceral adipose tissue weight as a function of animal groups on specified diets. Diets, sample collection, and statistical measures were as for FIG. 14A.

FIG. 15 shows F1 reduces systemic inflammation in DIO mice. Detection of cytokines and chemokines in the serum of animal groups on specified diets using membrane-based immunoassays. LD: lean diet (left two panels); HFD: high-fat diet (middle two panels); HFD+F1: high-fat diet supplemented with F1 (right two panels). Representative data from two mice per diet group were presented.

### DETAILED DESCRIPTION

The present disclosure relates to novel compositions of phytonutrients and methods of treating obesity by administering these compositions to subjects in need thereof. The compositions described herein are rationally designed compositions of phytonutrients that interfere with fat cell differentiation, a process commonly known as “adipogenesis”, to prevent weight gain and improve glycemic control. Phytonutrients are natural compounds in plants and mushrooms that have beneficial effects on human health. Phytonutrients have been proven to have anti-obesity effects, such as appetite reduction, modulation of lipid absorption and metabolism, enhancement of insulin sensitivity, thermogenesis and changes to the gut microbiota [6]. Consumption of phytonutrients is generally considered as a safe, widely available and inexpensive approach to prevent obesity and associated conditions.

The compositions include formulation of rationally combined phytonutrients for obesity prevention. The phytonutrients are rationally combined based on their complementary effects on the expression level of six adipogenic biomarker proteins. The formulation of rationally combined phytonutrients exhibit unique anti-adipogenic properties that are distinct from those of individual phytonutrients. In addition, the formulation of rationally combined phytonutrients is more potent and longer lasting than those of individual phytonutrients for the suppression of adipogenesis in cell cultures. The formulations of rationally combined phytonutrients disclosed herein may prevent weight gain, improve glucose tolerance, reduce blood triglyceride and LDL cholesterol, reduce liver steatosis and visceral adiposity, and/or reduce the level of inflammatory cytokines and chemokines in a subject's blood. Collectively, the formulation of rationally combined phytonutrients described herein may prevent weight gain, improve glycemic control, reduce blood lipid level, suppress liver steatosis, and/or reduce systemic inflammation, thus, lowering the risks of developing obesity-associated disease.

| Phytonutrient | Name | Chemical Structure |
|---|---|---|
| C1 | Berberine | 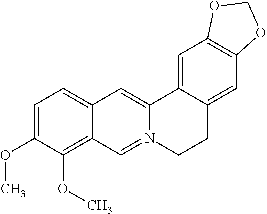 [SMILES: COc1ccc2cc3[n+](cc2c1OC)CCc1cc2c(cc1-3)OCO2] |
| C2 | Luteolin | 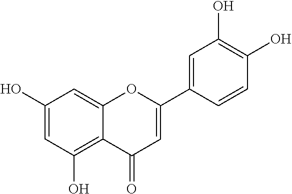 [SMILES: O=c1cc(-c2ccc(O)c(O)c2)oc2cc(O)cc(O)c12] |
| C3 | Resveratrol | 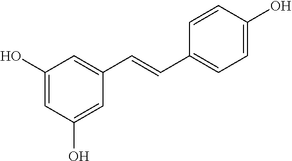 [SMILES: Oc1ccc(/C=C/c2cc(O)cc(O)c2)cc1] |
| C4 | Fisetin | 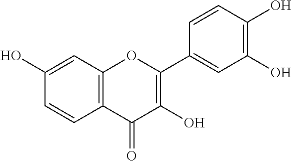 [SMILES: O=c1c(O)c(-c2ccc(O)c(O)c2)oc2cc(O)ccc12] |
| C5 | Quercetin | 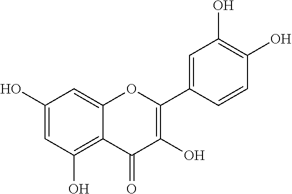 [SMILES: O=c1c(O)c(-c2ccc(O)c(O)c2)oc2cc(O)cc(O)c12] |
| C6 | Fucoidan | 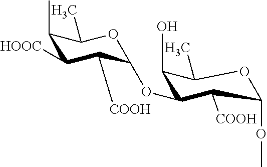 |
| C7 | Epigallocatechin Gallate (EGCG) | 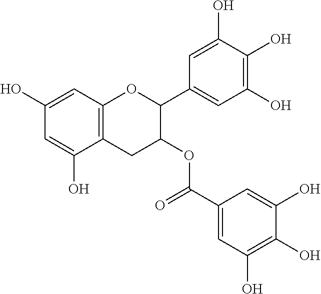 [SMILES: O=C(OC1Cc2c(O)cc(O)cc2OC1c1cc(O)c(O)c(O)c1)c1cc(O)c(O)c(O)c1] |
| C8 | Hesparidin | 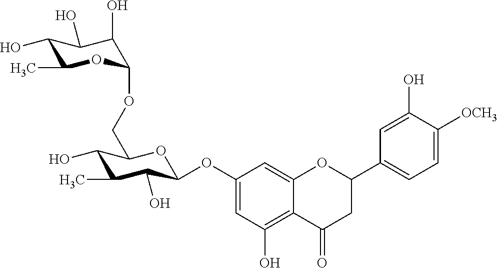 |
| C9 | Curcumin | 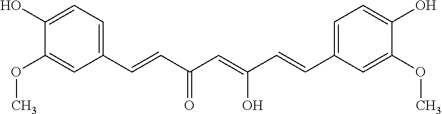 [SMILES: COc1cc(/C=C/C(=O)/C=C(O)/C=C/c2ccc(O)c(OC)c2)ccc1O] |
| F1 | A combination | All of the above |
| | of C1-C9 | |

In one aspect, the compositions disclosed herein may include any combination of phytonutrients C1, C2, C3, C4, C5, C6, C7, C8, and C9. For example, the combination of phytonutrients may be (1) C1, C2, C3, and C5; (2) C1, C2, C3, and C7; (3) C1, C3, C4, and C5; or C1, C3, C4, and C7. In a particular embodiment, the composition may include all nine phytonutrients C1, C2, C3, C4, C5, C6, C7, C8, and C9.

The ratio of individual components may vary, for example in an embodiment comprising all nine phytonutrients, C1 may account for about 18% (e.g., about 15%, 16%, 17%, 18%, 19%, 20%, 21%) of the formulation, C2 may account for about 10% (e.g., about 7%, 8%, 9%, 10%, 11%, 12%, 13%) of the formulation, C3 may account for about 18% (e.g., about 15%, 16%, 17%, 18%, 19%, 20%, 21%) of the formulation, C4 may account for about 9% (e.g., about 6%, 7%, 8%, 9%, 10%, 11%, 12%) of the formulation, C5 may account for about 9% (e.g., about 6%, 7%, 8%, 9%, 10%, 11%, 12%) of the formulation, C6 may account for about 9% (e.g., about 6%, 7%, 8%, 9%, 10%, 11%, 12%) of the formulation, C7 may account for about 9% of the formulation, C8 may account for about 9% (e.g., about 6%, 7%, 8%, 9%, 10%, 11%, 12%) of the formulation, and C9 may account for about 9% (e.g., about 6%, 7%, 8%, 9%, 10%, 11%, 12%) of the formulation. In some formulations, the ratio of C1:C2:C3:C4:C5:C6:C7:C8:C9 is about 2:1:2:1:1:1:1:1:1.

In another aspect, the invention relates to methods of treating or preventing obesity comprising administering a therapeutically effective amount of the compositions disclosed herein.

The compositions disclosed herein may improve glycemic control, reduce blood lipid level, suppress liver steatosis, reduce systemic inflammation, and/or lower the risk of developing obesity-associated disease.

### EXAMPLES

### Example 1: Screening for Anti-Adipogenic Properties

Using advanced proteomic methods, we screened hundreds of phytonutrients for their anti-adipogenic properties and identified nine phytonutrients (C1-C9) that exhibit complementary effects. We combined these nine phytonutrients into a formulation called F1 to synergize their anti-adipogenic effects. Similar to the beneficial synergistic interactions among multiple ingredients in botanical extracts, we anticipate that the interactions of nine phytonutrients provide synergistic multitargeted effects and neutralize the adverse side effects of individual phytonutrients. In cultures of human primary preadipocytes, we demonstrated that F1 was a much more potent and longer-lasting inhibitor of adipogenesis than individual phytonutrients. In an animal model of diet-induced obesity, we showed that F1 was effective at preventing weight gain, improving glucose tolerance, suppressing liver steatosis, and reducing visceral adiposity, blood lipids and systemic inflammation. Our experimental approaches, methods, conditions, and supporting data are presented in the following sections.

### Example 2: Function and Expression Analysis

Obesity is characterized by increased adipose tissue mass via hypertrophy, an increase in size of existing fat cells or adipocytes, or hyperplasia, the formation of new adipocytes from precursor cells or preadipocytes [7]. Adipogenesis is the process by which preadipocytes cells commit to the adipogenic lineage, express adipogenic genes and proteins, accumulate intracellular lipid storage and become fully differentiated adipocytes. Our approach toward obesity prevention was to identify phytonutrients that interfere with the expression of six following adipogenic biomarker proteins: PPARγ, SREBP1c, FASN, PLIN1, FABP4 and β-catenin. The function and expected expression level of these biomarker proteins during adipogenesis are listed in Table 1.

TABLE 1
Name, Function, and Expected Expression Level of Adipogenic Biomarker Proteins

| | | | Expression Level |
|---|---|---|---|
| Biomarker | | | During |
| Protein | Name | Function | Adipogenesis |
| PPARγ | Peroxisome | A transcription factor that promotes | Increase |
| | proliferator- | fat cell differentiation by regulating | |
| | activated receptor | glucose metabolism and lipid uptake | |
| | gamma | and storage. | |
| SREBP1c | Sterol regulatory | A transcription factor that regulates | Increase |
| | element-binding | glycolysis and sterol biosynthesis. | |
| | transcription factor | | |
| | 1c | | |
| FASN | Fatty acid synthase | An enzyme that catalyzes de novo | Increase |
| | | fatty acid synthesis | |
| PLIN1 | Perilipin 1 | A lipid droplet-associated protein that | Increase |
| | | regulates lipid storage. | |
| FABP4 | Fatty acid binding | Macrophage- and adipocyte-specific | Increase |
| | protein 4 | fatty acid binding protein that | |
| | | transport fatty acids. | |
| β-catenin | β-catenin | A protein that regulates cell-cell | Decrease |
| | | adhesion and gene expression | |

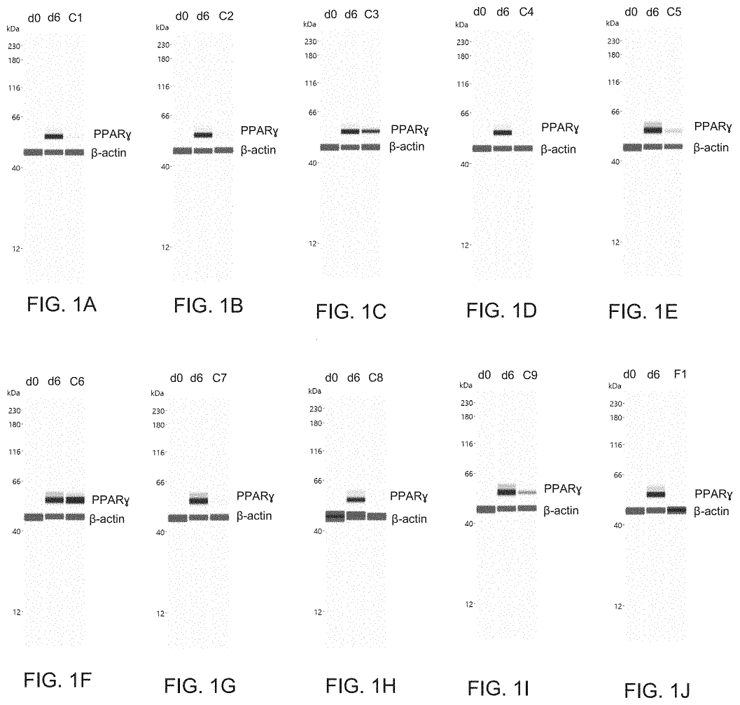

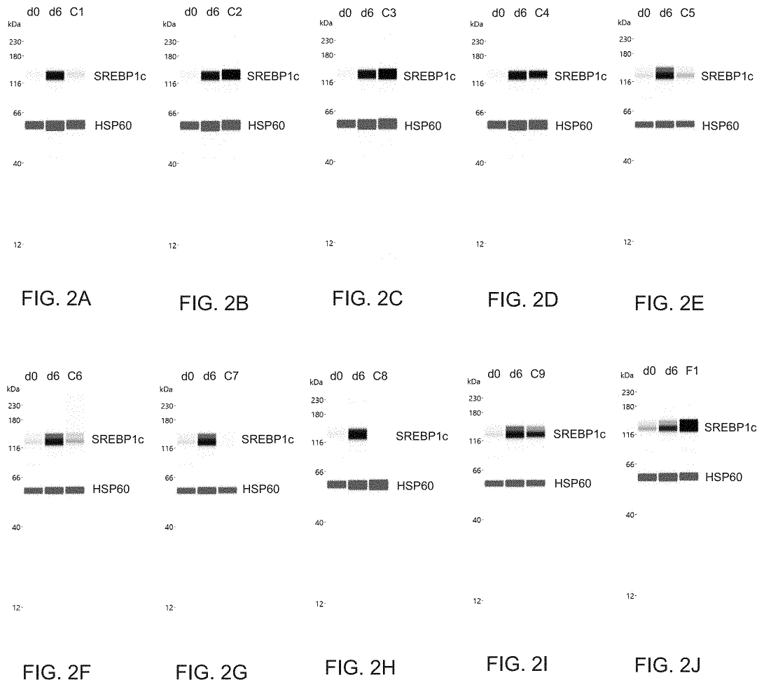

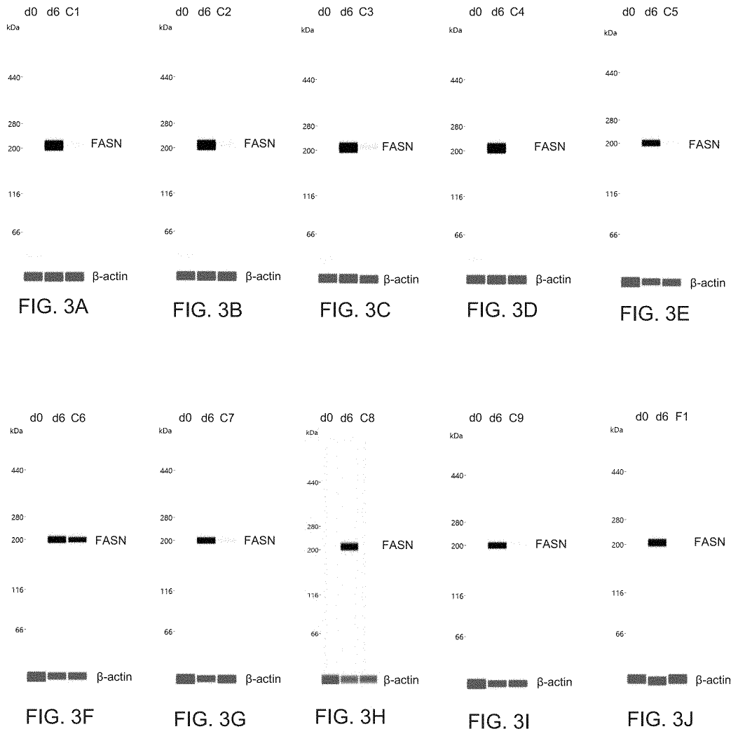

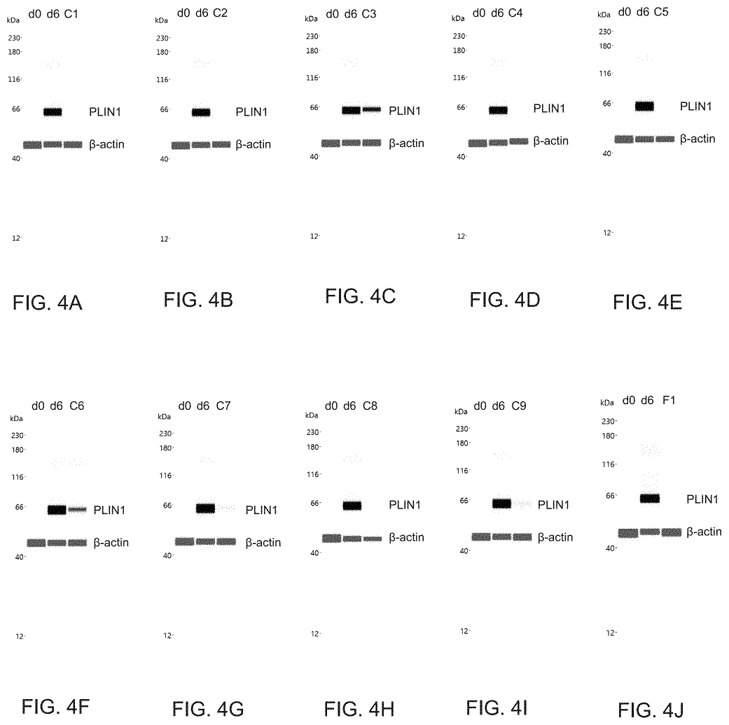

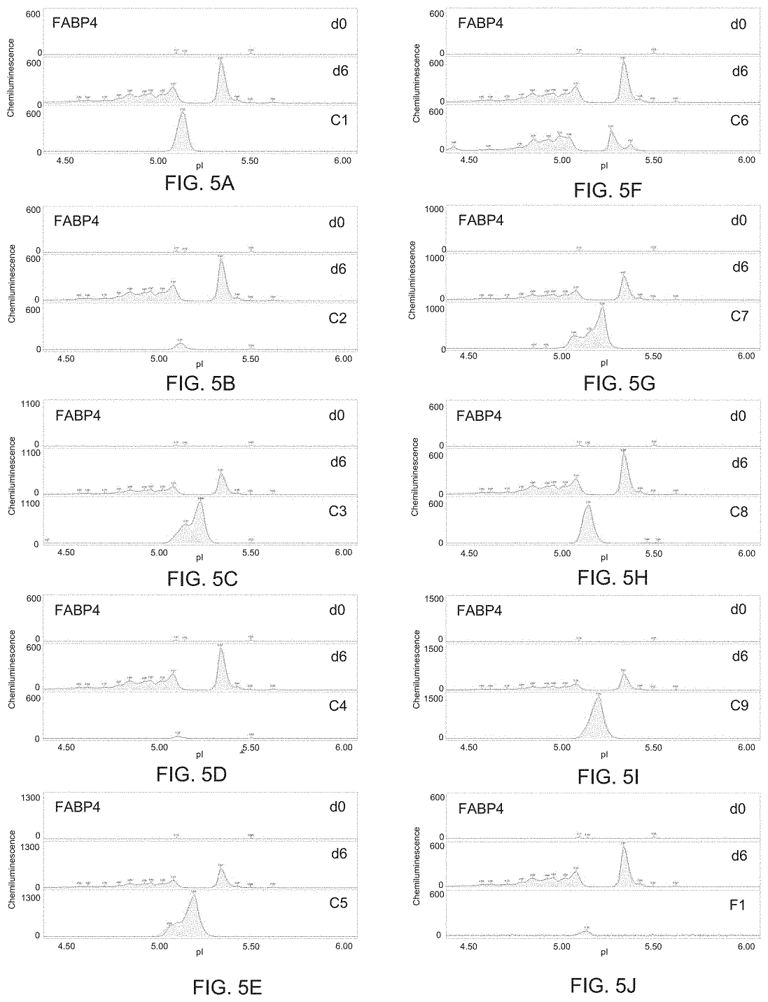

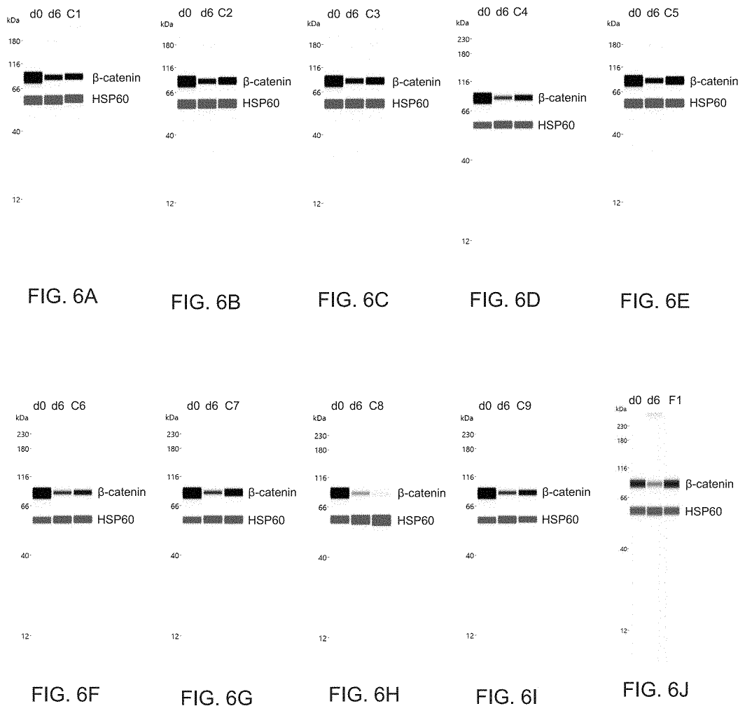

Briefly, preadipocytes grown to 2 days post-confluence were induced for adipogenesis via the addition of complete differentiation media for 6 days. On day 7, complete differentiation media were replaced with maintenance media and differentiation was allowed to continue until day 14. Phytonutrients were supplemented to the complete differentiation media to screen for their anti-adipogenic effects. Out of hundreds of phytonutrients screened, nine phytonutrients (C1-C9) were selected that had complementary effects on the expression level of six adipogenic biomarker proteins aforementioned. The effects of C1-C9 on the expression level of six biomarker proteins during adipogenesis are presented in FIGS. 1A-1J, FIGS. 2A-2J, FIGS. 3A-3J, FIGS. 4A-4J, FIGS. 5A-5J, FIGS. 6A-6J and summarized in Table 2.

TABLE 2
Expression Level of Adipogenic Biomarker Proteins
in the Presence of Phytonutrients

| Biomarker Protein | d0 | d6 | C1 | C2 | C3 | C4 | C5 | C6 | C7 | C8 | C9 | F1 |
|---|---|---|---|---|---|---|---|---|---|---|---|---|
| PPARγ | − | + | − | − | + | − | − | + | − | − | − | − |
| SREBP1c | − | + | − | + | + | + | − | − | − | − | + | + |
| FASN | − | + | − | − | − | − | − | + | − | − | − | − |
| PLIN1 | − | + | − | − | + | − | − | − | − | − | − | − |
| FABP4 | − | + | + | − | + | − | + | + | + | + | + | − |
| β-catenin | + | − | − | − | − | − | + | − | + | − | − | + |

The identities, chemical structures of C1-C9 and their half-maximal effective concentrations (EC₅₀) for the suppression of adipogenesis are listed in Table 3.

TABLE 3
Composition and Half-Maximal Effective Concentration of F1

| | | | | Composition in |
|---|---|---|---|---|
| Phytonutrient | Name | Chemical Structure | EC₅₀ | F1 |
| C1 | Berberine | 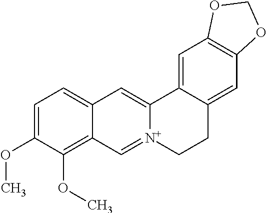 [SMILES: COc1ccc2cc3[n+](cc2c1OC)CCc1cc2c(cc1-3)OCO2] | 10 μM or 3.5 μg/ml | 18% |
| C2 | Luteolin | 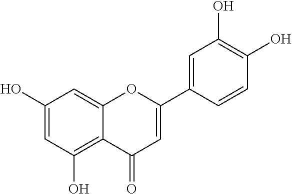 [SMILES: O=c1cc(-c2ccc(O)c(O)c2)oc2cc(O)cc(O)c12] | 20 μM or 5.7 μg/ml | 10% |
| C3 | Resveratrol | 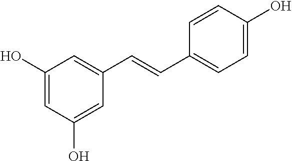 [SMILES: Oc1ccc(/C=C/c2cc(O)cc(O)c2)cc1] | 40 μM or 9.1 μg/ml | 18% |
| C4 | Fisetin | 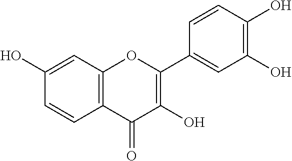 [SMILES: O=c1c(O)c(-c2ccc(O)c(O)c2)oc2cc(O)ccc12] | 50 μM or 14.3 μg/ml | 9% |
| C5 | Quercetin | 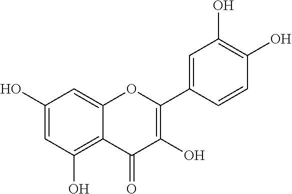 [SMILES: O=c1c(O)c(-c2ccc(O)c(O)c2)oc2cc(O)cc(O)c12] | 25 μM or 7.6 μg/ml | 9% |
| C6 | Fucoidan | 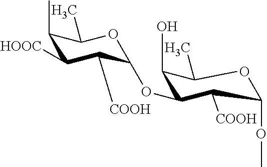 | 100 μg/ml | 9% |
| C7 | Epigallocatechin Gallate (EGCG) | 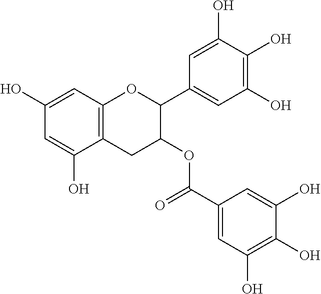 [SMILES: O=C(OC1Cc2c(O)cc(O)cc2OC1c1cc(O)c(O)c(O)c1)c1cc(O)c(O)c(O)c1] | 20 μM or 9.2 μg/ml | 9% |
| C8 | Hesparidin | 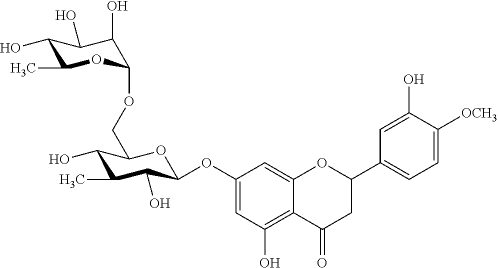 | 20 μM or 12.2 μg/ml | 9% |
| C9 | Curcumin | 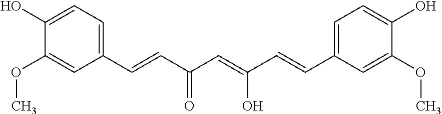 [SMILES: COc1cc(/C=C/C(=O)/C=C(O)/C=C/c2ccc(O)c(OC)c2)ccc1O] | 20 μM or 7.5 μg/ml | 9% |
| F1 | A combination | All of the above | 10 μg/ml | 100% |
| | of C1-C9 | | | |

*EC₅₀ indicates the experimentally determined half-maximal effective concentration for the suppression of adipogenesis in cultures of human primary preadipocytes.

Table legend: d0: preadipocytes in growth media; d6: differentiating adipocytes on the sixth day post-differentiation induced by complete differentiation media; C1-C9: differentiating adipocytes on the sixth day post-differentiation induced by complete differentiation media supplemented with individual C1-C9 phytonutrients; F1: differentiating adipocytes on the sixth day post-differentiation induced by complete differentiation media supplemented with F1; −: low expression level; +: high expression level.

### Example 3: Rationally Designed Formulations

Based on the complementary effects of C1-C9 on the expression level of six biomarker proteins, we aimed to synergize their anti-adipogenic properties via rational combinations. The three must-have objectives for the rationally designed formulations were: (1) suppression of the expression of lipogenic genes, de novo fatty acid biosynthesis, formation of lipid droplets, and fatty acids transport via negative regulation of PPARγ, FASN, PLIN1, and FABP4 expression, respectively; (2) activation of glycolysis via positive regulation of SREBP1c expression; and (3) preservation of cell-cell adhesion via inhibition of β-catenin degradation. The rationally designed formulations aimed to maintain the capability for glucose uptake and utilization of differentiating adipocytes while suppressing their capability for de novo fatty acid biosynthesis, lipid droplet formation, fatty acid transport, and morphological transformation from spindle to round shape.

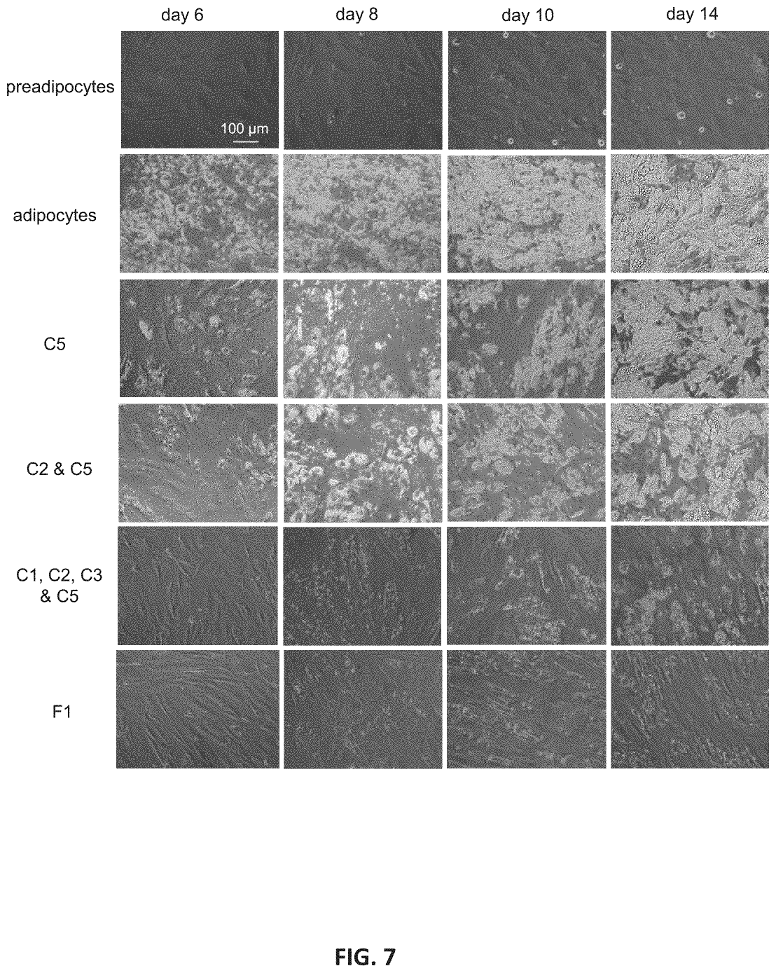

Based on the complementary effects of phytonutrients on six protein biomarkers summarized in Table 2, the following four possible combinations should theoretically meet the proposed design objectives: (1) C2 & C5, (2) C2 & C7, (3) C4 & C5, and (4) C4 & C7. The combinations of two phytonutrients were inadequate for prolonged suppression of adipogenesis (FIG. 7). Similar to individual phytonutrients, the combinations of two nutrients were able to suppress cytoplasmic lipid droplet accumulation by up to 60% during the first six days post-differentiation, where phytonutrients were present in the complete differentiation media. However, following their removal on day 7 via the replacement of complete differentiation media with maintenance media, cytoplasmic lipid droplet accumulation immediately resumed. On day 14, it was not possible to differentiate cell cultures that were previously treated with complete differentiation media alone versus those that were treated with complete differentiation media supplemented with either individual phytonutrients or combinations of two phytonutrients. Clearly, individual phytonutrients or combinations of two phytonutrients did not have lasting anti-adipogenic effects following their removal from the differentiating cell cultures.

Next, the number of phytonutrients in the combinations was increased and reported substantial improvement in both anti-adipogenic potency and duration with the addition of both C1 and C3 compounds to the existing combinations of two phytonutrients. The combinations of four phytonutrients included the following: (1) C1, C2, C3, & C5; (2) C1, C2, C3, & C7; (3) C1, C3, C4, & C5; and (4) C1, C3, C4, & C7. These combinations of four phytonutrients were able to suppress cytoplasmic lipid droplet accumulation by more than 90% during the first six days of differentiation and up to 50% on the 14^{th} day post-differentiation (FIG. 7). Without wishing to be bound by theory, it is plausible that additional phytonutrients added necessary redundancy and increased anti-adipogenic potency of the combinations. In addition, phytonutrients are likely to affect the expression of biomarker proteins via different pathways. Combinations of four or more phytonutrients introduced sufficient blockades to adipogenesis, which persisted even after the removal of phytonutrients from the culturing media.

### Example 4: Formula F1—A Highly Potent and Long-Lasting Inhibitor of Adipogenesis

The combination of all nine phytonutrients (C1-C9) was the most effective for the suppression of adipogenesis. The formulation comprising all nine phytonutrients was named F1. Formulation F1 met all of the must-have design objectives including negative regulation of the expression of PPARγ, FASN, PLIN1, and FABP4; positive regulation the expression of SREBP1c; and preservation of the expression level of β-catenin in differentiating adipocytes (FIGS. 1A-1J, FIGS. 2A-2J, FIGS. 3A-3J, FIGS. 4A-4J, FIGS. 5A-5J, FIGS. 6A-6J and Table 2). The half-maximal effective concentration of F1 for the suppression of adipogenesis and the composition of F1 are listed in Table 3.

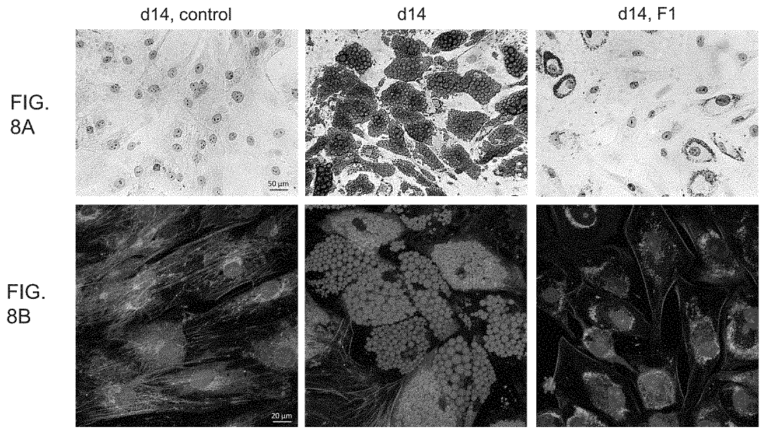

Most importantly, F1 was a highly effective inhibitor of adipogenesis (FIG. 7). F1 supplementation suppressed cytoplasmic lipid accumulation by 100% during the first 6 days of adipogenesis and by more than 95% on the 14^{th} day post-differentiation. In addition, F1 supplementation allowed the maintenance of the spindle shape of differentiating cells through two weeks of differentiation. High-resolution images obtained with brightfield and confocal microscopy to show the ability of F1 to suppress cytoplasmic lipid droplet accumulation in differentiating adipocytes at fourteenth day post-differentiation are presented in FIGS. 8A and 8B. Taken together, F1 was a highly potent and long-lasting inhibitor of adipogenesis in cell cultures of human primary preadipocytes.

### Example 5: F1 Prevented Weight Gain in a Diet-Induced Obesity (DIO) Mouse Model

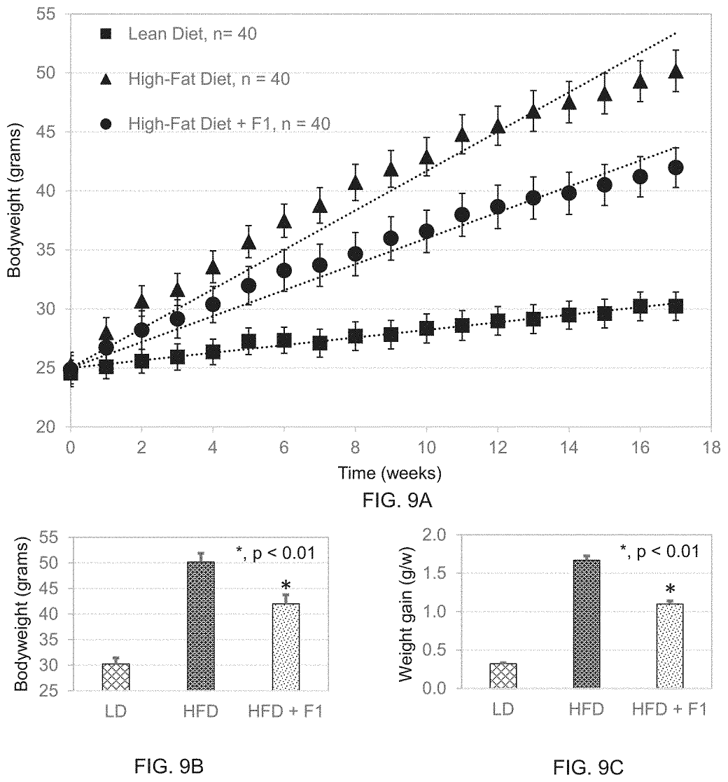

The therapeutic effects of F1 for obesity prevention were evaluated using a DIO mouse model (FIGS. 9A-9C). Mice were divided into three groups: a group of 40 mice were fed with a lean diet, a group of 40 mice were fed with a high-fat diet, and a group of 40 mice were fed with a high-fat diet supplemented with F1. F1 was supplemented to ground pellets at 0.1% by weight, leading to an approximately daily dose of 200 mg/kg for mice, or approximately 16 mg/kg of human equivalent dose. All mice were placed on their respective diets for 17 weeks. At the beginning of the experiments, all mice were male, approximately 10 weeks old, and had bodyweights of approximately 25 grams. Mice fed with a lean diet gained weight at a steady rate of 0.3 gram per week and reached an average of 30 grams in bodyweight after 17 weeks. Mice fed with a high-fat diet gained weight rapidly at a rate of 1.7 grams per week and reached an average of 50 grams in bodyweight after 17 weeks. Interestingly, mice fed with a high-fat diet supplemented with F1 gained weight a rate of 1.1 grams per week and reached an average of 42 grams in bodyweight after 17 weeks. On average, F1 supplementation reduced weight gain of DIO mice by approximately 45%.

### Example 6: F1 Improved Glucose Tolerance in DIO Mice

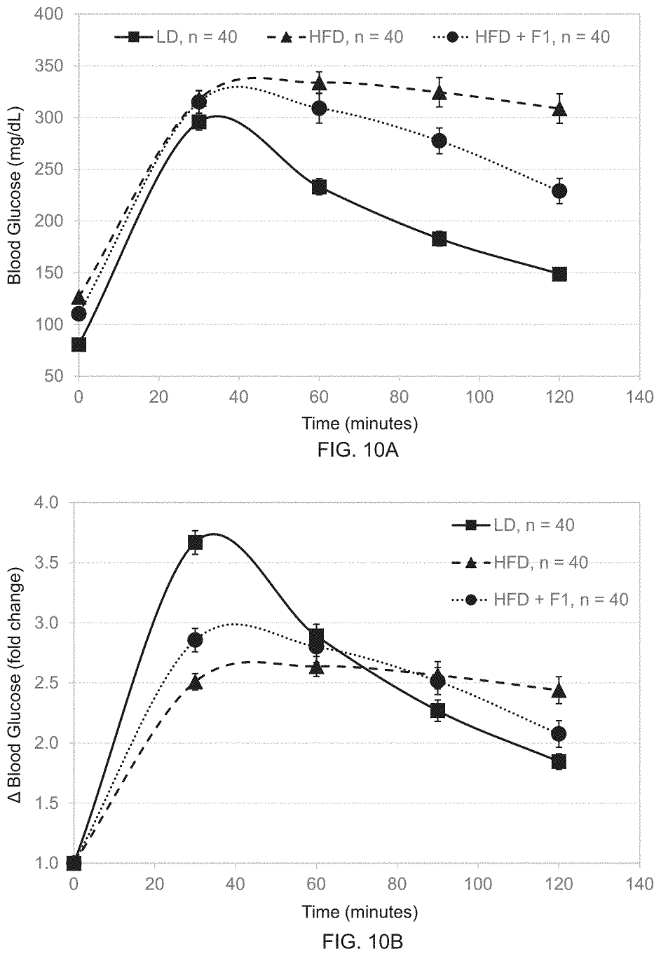

At 16^{th} week, glucose tolerance tests were performed for all mice after 16 hours of overnight fasting (FIGS. 10A and 10B). Fasting blood glucose levels were 81 mg/dL, 127 mg/dL, and 110 mg/dL for mice fed with a lean diet, a high-fat diet, and a high-fat diet supplemented with F1, respectively. Following intraperitoneal injection of 20% glucose at 2 g of glucose per kg of body mass, blood samples were collected via the tail veins at 0, 30, 60, 90, and 120 minutes and measured for blood glucose levels using a glucometer. Mice fed with a lean diet showed strong ability to dynamically regulate blood glucose level. In mice fed with a lean diet, blood glucose increased by nearly 4 folds, peaked at around 30 minutes post-injection, and steadily declined to slightly less than 2 folds higher than the baseline blood glucose level at 120 minutes post-injection. In contrast, mice fed with a high-fat diet were unable to regulate blood glucose level. In mice fed with a high-fat diet, blood glucose increased by 2.5 folds at 30 minutes post-injection, and stayed elevated at around 2.5 folds higher than the baseline blood glucose level until 120 minutes post-injection. Interestingly, mice fed with a high-fat diet supplemented with F1 had better control of blood glucose level compared to mice fed with a high-fat diet alone. Following glucose injection, blood glucose increased by 3 folds at 30 minutes post-injection, and steadily declined to around 2 folds higher than the baseline blood glucose level at 120 minutes post-injection. F1 supplementation clearly improved glucose tolerance in DIO mice.

### Example 7: F1 Reduced Blood Lipids in DIO Mice

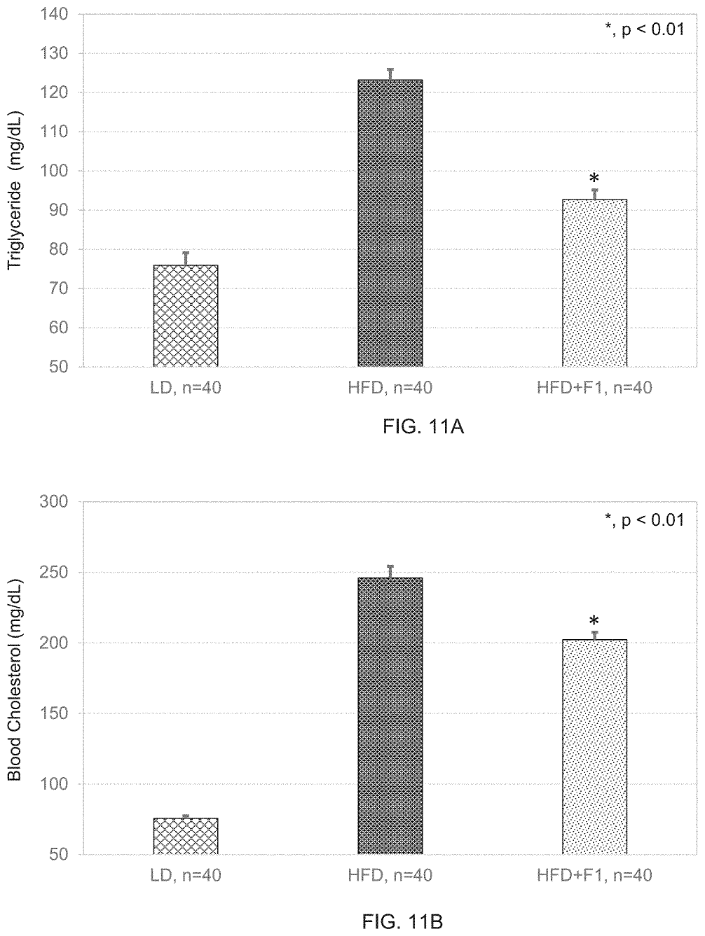

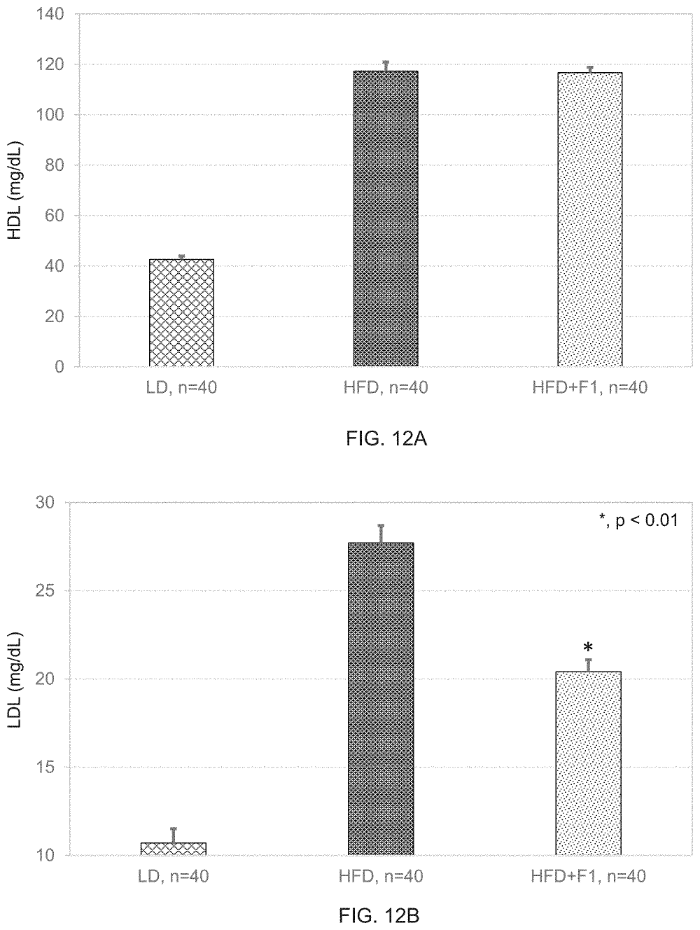

At 17^{th} week, blood and tissue samples were terminally collected from all mice in this study. Blood samples were sent to IDEXX Analytics (West Sacramento, Calif.) for measurement of triglyceride, total cholesterol, high-density lipoprotein (HDL) cholesterol, and low-density lipoprotein (LDL) cholesterol. The average triglyceride levels were 76 mg/dL, 123 mg/dL, and 93 mg/dL for mice fed with a lean diet, a high-fat diet, and a high-fat diet supplemented with F1, respectively (FIG. 11A). The average total cholesterol levels were 76 mg/dL, 246 mg/dL, and 202 mg/dL for mice fed with a lean diet, a high-fat diet, and a high-fat diet supplemented with F1, respectively (FIG. 11B). The average HDL cholesterol levels were 43 mg/dL, 117 mg/dL, and 117 mg/dL for mice fed with a lean diet, a high-fat diet, and a high-fat diet supplemented with F1, respectively (FIG. 12A). The average LDL cholesterol levels were 11 mg/dL, 28 mg/dL, and 20 mg/dL for mice fed with a lean diet, a high-fat diet, and a high-fat diet supplemented with F1, respectively (FIG. 12B). The blood lipid profiles clearly indicated that F1 supplementation reduced blood triglyceride and LDL cholesterol in DIO mice.

### Example 8: F1 Reduced Liver Steatosis and Visceral Adiposity in DIO Mice

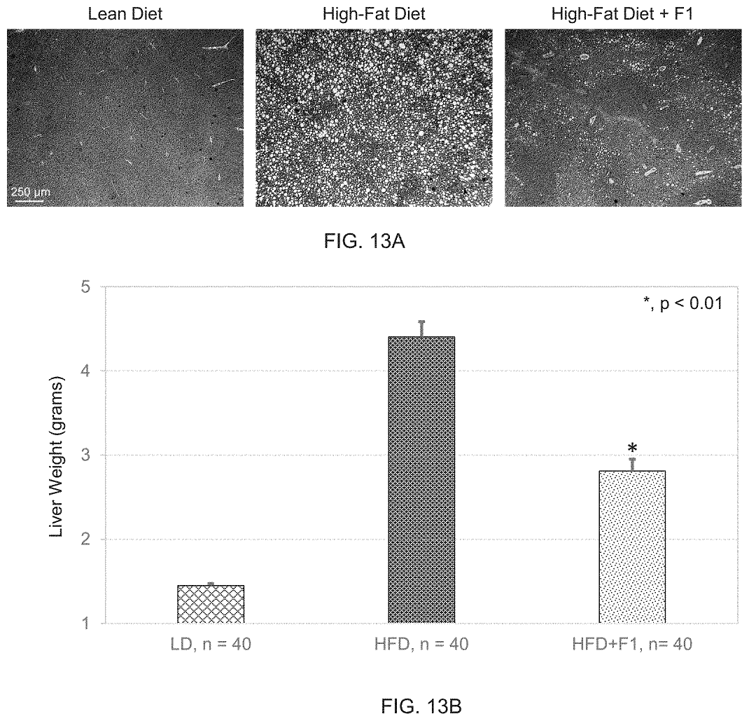

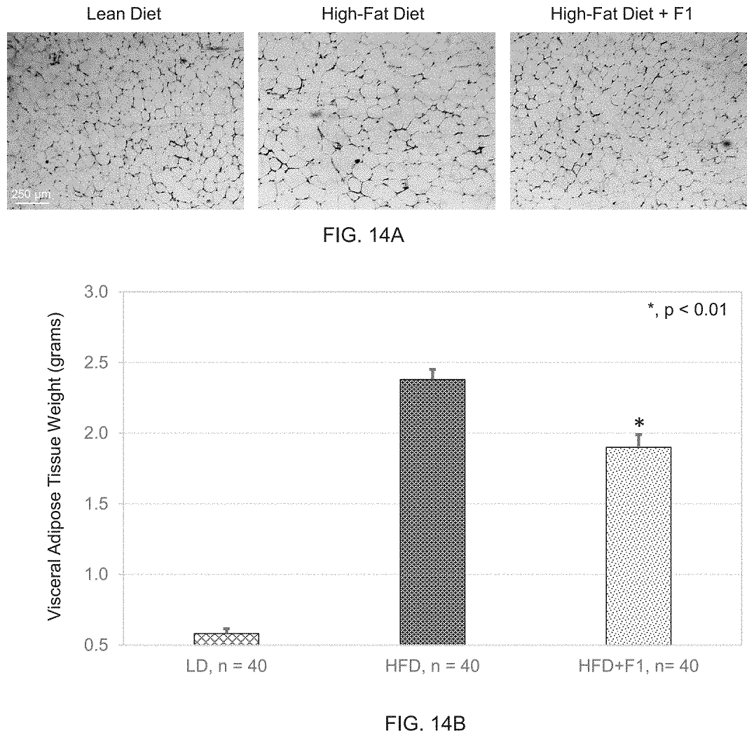

Terminally collected liver and visceral adipose tissues were sent to IHC World (Woodstock, Md.) for hematoxylin & eosin (H&E) histology preparation. H&E histology revealed a complete absence of any lipid droplet accumulation in liver tissues of mice fed with a lean diet, severe lipid droplet accumulation in liver tissues of mice fed with a high-fat diet, and substantially reduced level of lipid droplet accumulation in liver tissues of mice fed with a high-fat diet supplemented with F1 compared to those of mice fed with a high-fat diet alone (FIG. 13A). On average, the liver weights were approximately 1.5 g, 4.4 g, and 2.8 g for mice fed with a lean diet, a high-fat diet, and a high-fat diet supplemented with F1, respectively (FIG. 13B). Furthermore, H&E histology revealed an average diameter of lipid droplets of visceral adipocytes that was 2 times higher for mice fed with a high-fat diet compared to those fed with a lean diet (FIG. 14A). On the other hand, the average diameter of lipid droplets of visceral adipocytes were 1.5 times higher for mice fed with a high-fat diet supplemented with F1 compared to those fed with a lean diet. Consistently, the average VAT weights were 0.58 g, 2.4 g, and 1.9 g for mice fed with a lean diet, a high-fat diet, and a high-fat diet supplemented with F1, respectively (FIG. 14B). H&E histology revealed that F1 reduced liver steatosis and visceral adiposity in DIO mice.

### Example 9: F1 Reduced Systemic Inflammation in DIO Mice

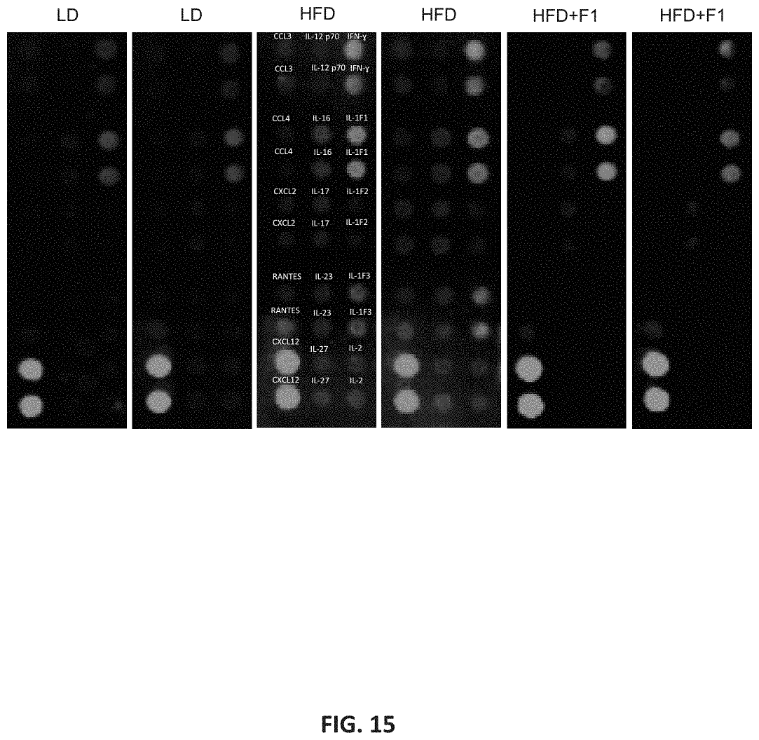

Obesity is associated with systemic low-grade chronic inflammation that increases the risks for the development of metabolic disease. Using a membrane-based immunoassay to measure an array of inflammatory chemokines and cytokines, substantial increases in the presence of chemokines (CCL3, CCL4, CXCL2, and RANTES) and cytokines (IL-1F2, IL-1F3, IL-2, IL-12p70, IL16, IL17, IL23, and IL27) were reported in the blood samples of mice fed with a high-fat diet compared to those of mice fed with a lean diet (FIG. 15). Interestingly, supplementation with F1 substantially reduced the inflammatory biomarkers in the blood samples of mice fed with a high-fat diet. The data indicated that F1 supplementation reduced systemic inflammation in DIO mice.

### Example 10: A Potent and Long-Lasting Formulation of Phytonutrients for Obesity Prevention

In summary, described herein is a formulation of rationally combined phytonutrients for obesity prevention. The phytonutrients were rationally combined based on their complementary effects on the expression level of six adipogenic biomarker proteins. The formulation of rationally combined phytonutrients had unique anti-adipogenic properties that were distinct from those of individual phytonutrients. In addition, the formulation of rationally combined phytonutrients was more potent and longer lasting than those of individual phytonutrients for the suppression of adipogenesis in cell cultures. Furthermore, in a DIO animal model, dietary supplementation with the formulation of rationally combined phytonutrients prevented weight gain, improved glucose tolerance, reduced blood triglyceride and LDL cholesterol, reduced liver steatosis and visceral adiposity, and reduced the level of inflammatory cytokines and chemokines in the blood. Collectively, the formulation of rationally combined phytonutrients described herein is capable of preventing weight gain, improving glycemic control, reducing blood lipid level, suppressing liver steatosis, and reducing systemic inflammation, thus, lowering the risks of developing obesity-associated disease.

### A. MATERIALS AND METHODS

### Adipogenesis assays

Primary human preadipocytes were isolated from subcutaneous adipose tissues of a single donor who was undergoing elective surgery. Preadipocytes were cultured and differentiated into adipocytes using a previously published protocol [8]. Briefly, preadipocytes were grown to confluence in growth media comprising Minimum Essential Medium a supplemented with 10% fetal bovine serum, 100 units/ml penicillin, and 100 μg/ml streptomycin. At 2 days post-confluence, growth media were aspirated off the culture dishes and complete differentiation media were added. Complete differentiation media comprise DMEM/F12 with 18.5 mM glucose, HEPES (15 mM), NaHCO3 (25 mM), 100 units/ml penicillin, 100 μg/ml streptomycin, d-biotin (33 μM), pantothenate (17 μM), dexamethasone (100 nM), insulin (100 nM), rosiglitazone (1 μM), IBMX (0.5 mM), triiodothyronine (T3, 2 nM), and transferrin (10 μg/ml). On day three post-differentiation, complete differentiation media were replenished. On day seventh post-differentiation, complete differentiation media were replaced with maintenance media. Maintenance media comprise DMEM/F12, 100 units/ml penicillin, 100 μg/ml streptomycin, HEPES (15 mM), NaHCO3 (25 mM), d-biotin, pantothenate, insulin (10 nM), and dexamethasone (10 nM). Maintenance media were replenished on days tenth post-differentiation. Complete differentiation of preadipocytes into adipocytes were achieved on day fourteenth post-differentiation.

### Cell Cultures for the Screening of Anti-Adipogenic Phytonutrients

Three cell cultures were generated for the screening of each phytonutrient: a cell culture of preadipocytes at 2 days post-confluence (d0), a cell culture of differentiating adipocytes at 6 days post-differentiation (d6), and cell culture of differentiating adipocytes at 6 days post-differentiation in the present of C1-C9 or F1. Phytonutrients were added to the complete differentiation media on day 0 and day 3 post-differentiation. The concentrations of C1-C9 and F1 are listed in Table 3.

### Preparation of Cell Lysates

Approximately one million cells were incubated on ice for 10 minutes with 60 μl of lysis buffer (cat. no. 040-764, ProteinSimple, Santa Clara, Calif., USA), sonicated 4 times for 5 seconds each, mixed by rotation for 2 hours at 4° C., and centrifuged at 12,000 rpm in an Eppendorf 5430R microfuge for 20 minutes at 4° C. The supernatant was collected as the cell lysate. The total protein concentration in the cell lysate was determined with a Bradford protein assay and adjusted to a final concentration of 0.3 μg/μl with separation gradients (cat. no. Premix G2, pH 5-8, ProteinSimple, Santa Clara, Calif.) for charge-based cIEF immunoassays or to 0.4 μg/μl with denaturing buffers (cat. no. PS-ST01EZ or PS-ST03EZ, ProteinSimple) for size-based Western immunoassays.

### Capillary Western Immunoassays

Cell lysates in denaturing buffers were denatured at 95° C. for 5 minutes, and then transferred to assay plates (cat. no. SM-W004 or SM-W008, ProteinSimple) preloaded with blocking reagents, wash buffer, primary and secondary antibodies, and chemiluminescent substrates. Sized-based protein separation and detection in capillaries were performed using the default protocols of the Jess system (ProteinSimple). β-Actin and HSP60 were used as loading controls. All capillary Western immunoassays were performed in triplicate for each protein, and duplicate experiments were performed for each treatment condition, producing six repeated measurements per protein. Expression levels of PPARγ, SREBP1c, FASN, PLIN1, and β-catenin were detected with capillary Western immunoassays.

### Capillary Isoelectric Focusing Immunoassays

Cell lysates in separation gradients were loaded into 384-well assay plates (cat. no. 040-663, ProteinSimple) preloaded with primary and secondary antibodies and chemiluminescent substrates. Charge-based protein separation and detection in individual capillaries were performed using the default protocols of the NanoPro 1000 system (ProteinSimple). Hsp70 was used as the loading control. All cIEF immunoassays were performed in triplicate for each protein, and duplicate experiments were performed for each treatment condition, producing six repeated measurements per protein. Expression level of FABP4 was detected with capillary isoelectric focusing immunoassays.

### Antibodies

The antibodies used to measure protein expression levels are listed in Table 4.

TABLE 4
List of Primary and Secondary Antibodies

| No | Antibody | Cat. No. | Vendor |
|---|---|---|---|
| 1 | PPARγ | CS2443 | Cell Signaling (Danvers, MA) |
| 2 | SREBP1c | NB600-582 | Novus Biologicals (Centennial, |
| 3 | FASN | CS3189 | Cell Signaling |
| 4 | PLIN1 | CS9349 | Cell Signaling |
| 5 | FABP4 | AB92501 | Abeam (Cambridge, MA) |
| 6 | β-catenin | NBPI- | Novus Biologicals |
| 7 | β-actin | MAB8929 | R&D Systems (Minneapolis, |
| 8 | HSP60 | F1800 | R&D Systems |
| 9 | HSP70 | 4872 | Cell Signaling |
| 10 | Secondary antibody (anti-rabbit | 040-656 | Protein Simple (Santa Clara, CA) |
| 11 | Secondary antibody (anti-rabbit | 042-206 | Protein Simple |
| 12 | Secondary antibody (anti-mouse | 042-205 | Protein Simple |
| 13 | Secondary antibody (anti-rabbit | 043-819 | Protein Simple |
| 14 | Secondary antibody (anti-mouse | 043-821 | Protein Simple |

### DIO Animal Model

C57BL/6J mice (male, ˜10 weeks old, Jackson Lab, Bar Harbor, Me.) were divided into three groups: a group of 40 mice fed with a lean diet, a group of 40 mice fed with a high-fat diet, and a group comprising mice fed with a high-fat diet supplemented with F1. The lean diet (cat. no. TD7001, Teklad Diets, Madison, Wis.) comprised protein (25.2% by weight), carbohydrate (39.5% by weight), fat (4.4% by weight), and others (30.9% by weight, ash, fibers, others). The lean diet has 3 kcal/g, with 34% of kcal from protein, 53% of kcal from carbohydrate, and 13% of kcal from fat. The high-fat diet (cat. no. TD88137, Teklad Diets) comprised protein (17.3% by weight), carbohydrate (48.5% by weight), fat (21.2% by weight), and others (13% by weight, ash, fibers, others). The high fat diet has 4.5 g/kcal, with 15.2% of kcal from protein, 42.7% of kcal from carbohydrate, and 42% of kcal from fat. F1 was supplemented at 0.1% by weight leading to an approximately daily dose of 200 mg/kg for mice, or approximately 16 mg/kg of human equivalent dose. Mice groups were placed on their respective diets in the form of ground pellets for 17 weeks. Glucose tolerance tests using standard protocols were performed at 16^{th} week. Terminal tissue and blood samples collection were performed at 17^{th} week. Collected liver and visceral adipose tissues were sent to IHC WORLD (Woodstock, Md.) for histopathology analysis. Collected blood samples were sent to IDEXX Analytics (West Sacramento, Calif.) for measurement of triglyceride, cholesterol, HDL, and LDL. The Proteome Profiler Mouse Cytokine Array Kits (cat. no. ARY006, R&D Systems, Minneapolis, Minn.) were used to measure inflammatory cytokines in collected blood samples. All animal studies were performed in conformity with the Public Health Service Policy on Humane Care and Use of Laboratory Animals and with the approval of the Animal Care and Use Committee at Roseman University of Health Sciences.

### REFERENCES

1. Hales, C. M.; Carroll, M. D.; Fryar, C. D.; Ogden, C. L. Prevalence of Obesity and Severe Obesity Among Adults: United States, 2017-2018. National Center for Health Statistics, 2020, Data Brief No. 360.
2. Hruby, A.; Hu, F. B. The Epidemiology of Obesity: A Big Picture. Pharmacoeconomics 2015, 33, 673-689.
3. National Institutes of Health. Clinical Guidelines on the Identification, Evaluation, and Treatment of Overweight and Obesity in Adults. NIH 1998, NIH PUBLICATION NO. 98-4083.
4. Van Gaal, L.; Scheen, A. Weight management in type 2 diabetes: current and emerging approaches to treatment. Diabetes Care 2015, 38, 1161-1172.
5. Kusminski, C. M.; Bickel, P. E.; Scherer, P. E. Targeting adipose tissue in the treatment of obesity-associated diabetes. Nat Rev Drug Discov 2016, 15, 639-660.
6. Martel, J.; Ojcius, D. M.; Chang, C. J.; Lin, C. S.; Lu, C. C.; Ko, Y. F.; Tseng, S. F.; Lai, H. C.; Young, J. D. Anti-obesogenic and antidiabetic effects of plants and mushrooms. Nat Rev Endocrinol 2017, 13, 149-160.
7. Ghaben, A. L.; Scherer, P. E. Adipogenesis and metabolic health. Nat Rev Mol Cell Biol 2019, 20, 242-258.
8. Lee, M. J.; Fried, S. K. Optimal protocol for the differentiation and metabolic analysis of human adipose stromal cells. Methods Enzymol. 2014, 538, 49-65.

1. A composition comprising a combination of phytonutrients selected from the group consisting of C1, C2, C3, C4, C5, C6, C7, C8, and C9.

2. The composition of claim 1, wherein said combination of phytonutrients comprises C1, C2, C3, and C5.

3. The composition of claim 1, wherein said combination of phytonutrients comprises C1, C2, C3, and C7.

4. The composition of claim 1, wherein said combination of phytonutrients comprises C1, C3, C4, and C5.

5. The composition of claim 1, wherein said combination of phytonutrients comprises C1, C3, C4, and C7.

6. The composition of claim 1, wherein said combination of phytonutrients comprises C1, C2, C3, C4, C5, C6, C7, C8, and C9.

7. The composition of claim 6, wherein the ratio of C1:C2:C3:C4:C5:C6:C7:C8:C9 is about 2:1:2:1:1:1:1:1:1.

8. The composition of claim 6, wherein C1 and/or C3 comprises about 18% of the composition respectively.

9. A method of treating or preventing obesity comprising administering a therapeutically effective amount of the composition of claim 1 to a subject in need thereof.

10. The method of claim 9, wherein said method improves glycemic control.

11. The method of claim 9, wherein said method reduces blood lipid level.

12. The method of claim 9, wherein said method suppresses liver steatosis.

13. The method of claim 9, wherein said method reduced systemic inflammation.

14. The method of claim 9, wherein the risk of developing obesity-associated disease is lowered.
